(19) United States
(12) Patent Application Publication
(10) Pub. No.: US 2018/0296182 A1
(43) Pub. Date: Oct. 18, 2018

(54) Apparatus And Method For Non-Invasive Determination Of Intracranial Pressure

(71) Applicants: Jonathan Maltz, Oakland, CA (US); Arminas Ragauskas, LT-51362 Kaunas (LT)

(72) Inventors: Jonathan Maltz, Oakland, CA (US); Arminas Ragauskas, LT-51362 Kaunas (LT)

(21) Appl. No.: 15/941,919

(22) Filed: Mar. 30, 2018

Related U.S. Application Data
(60) Provisional application No. 62/479,645, filed on Mar. 31, 2017.

(51) Int. Cl. A61B 8/08; A61B 5/03; A61B 5/00; A61B 8/00

(52) U.S. Cl. CPC A61B 8/0808; A61B 5/031; A61B 5/0053; A61B 5/7246; A61B 8/461; A61B 8/4209; A61B 8/488

(57) ABSTRACT

An apparatus and method for noninvasively measuring intracranial pressure of a subject using an ultrasound transducer. The transducer is used to measure arterial wall movement of an intracranial segment and an extracranial segment of the subject's ophthalmic artery as different external pressure forces are applied to the orbital area of the subject. When the waveforms of arterial wall movement between the intracranial segment and the extracranial segment are similar the intracranial pressure can be determined.

### CROSS-REFERENCE TO RELATED APPLICATIONS

This patent application claims the benefit under Title 35 United States Code, 119(e), U.S. Provisional Patent Application No. 62/479,645, filed Mar. 31, 2017.

### FIELD OF THE INVENTION

The invention relates noninvasive measurement of intracranial pressure, and more specifically to a system and methods for noninvasive measurement and monitoring of intracranial pressure using ultrasound transducers.

### BACKGROUND OF THE INVENTION

U.S. Pat. No. 5,951,477 (the '477 patent) teaches how intracranial pressure (ICP) can be measured non-invasively by comparing blood flow velocities in the intra-cranial and extra-cranial segments of the ophthalmic artery (IOA and EOA, respectively). External pressure is exerted on the orbit of the eye using an inflatable air cuff until the flow characteristics in the IOA and EOA are equalized. A major advantage of the method described is that is produces a calibrated measure of ICP.

There are potential deficiencies in the existing methods for measuring intracranial pressure. In some cases, the quality of the Doppler flow signals obtained is not sufficient to allow determination of ICP. This is because the signal-to-noise ratio (SNR) of the acquired signals is too low. Reasons for this can be attributed to several factors. First, the IOA segment lies at a depth of 6-8 cm. Ultrasound is attenuated with distance. Thus, the ultrasound is thus required to traverse 12-16 cm, which can result in a large amount of attenuation.

Second, Doppler flow signals are 45-60 dB lower in power than wall reflection signals, which inherently leads to a low SNR. Third, the FDA and other regulations limit the amount of power that can be used for ultrasound ophthalmic studies. This limits the maximum attainable SNR. Fourth, the ophthalmic artery has a diameter of approximately 1.5 mm. The artery runs approximately parallel to the ultrasound beam. This means that the artery is difficult to locate, and the size of the ultrasound gate rate is limited, which limits SNR.

Fifth, when ultrasound traverses fluid-filled regions such as the eye, reflections and reverberations occur. This ultrasound clutter can obscure the flow velocity signals, especially during the lower flow diastolic phase.

It is therefore desired to provide noninvasive measurement of intracranial pressure with higher quality signals, in that the detected wave reflections exhibit inherently higher SNR. Improved signal fidelity allows for the detection of lower amplitude waveform features.

Further, the quality of Doppler signals depends on the location of the Doppler range gate in the vessel. It is dependent on the position of the range gate within the lumen, and also the angle between the insonation line and the flow direction. It is further desired to provide a method and system that has higher SNR signals that can better tolerate suboptimal range gate location and insonation line selection.

What is also needed is higher signal quality that translates, practically, to higher precision of the determined ICP, higher accuracy, higher repeatability, and fewer measurement datasets that must be discarded owing to poor signal quality. Further, it is desired that measurements can also be performed faster, since fewer pulses are needed for analysis.

It is also desired in the field to use measurement of a primary quantity rather than a secondary quantity. When external pressure is applied to the EOA, this changes the local elastic properties, making the vessel wall more elastically compliant. What is needed in a method and apparatus that recognizes this is the primary effect of the variable applied pressure used to determine ICP and takes direct measurements to be used to calculate ICP. In general, it is preferred to measure a primary affected property rather than a secondary, derived, property.

What is also needed is an apparatus and method that makes it easier to position the transducer and range gates in order to acquire the required signals.

### SUMMARY OF THE INVENTION

Accordingly, it is an object of the present invention to provide an apparatus and method for a method for noninvasively measuring intracranial pressure (ICP) non-invasively that is accurate, has high SNR signals and measures a primary affected property.

It is a further object of the present invention to provide a method that makes it easier to position the transducer and range gates. Another object of the invention is to provide a method and apparatus with higher signal quality that translates, practically, to higher precision of the determined ICP, higher accuracy, higher repeatability, and fewer measurement datasets that must be discarded owing to poor signal quality. Further, it is an object of the invention to allow for measurements to be more quickly taken since fewer pulses are needed for analysis.

These and other objectives are achieved by providing a system for a method for noninvasively measuring intracranial pressure (ICP) of a subject based on comparing the motion of the arterial wall, in two or more arterial segments, one segment being substantially subject to, and one substantially not subject to, intracranial pressure.

In some embodiments, ICP is measured by comparing the motion of the arterial wall and adjacent tissues. In some embodiments the motion of the arterial wall and adjacent tissues is assessed using ultrasound. In some embodiments, ultrasound signals are used to determine wall motion waveform features derived from at least two segments of an ophthalmic artery, and in which the similarity of wall motion waveform features, between waveforms derived from the at least two segments of the ophthalmic artery, as a function of external pressure applied to the orbit of the eye of subject, determines the estimate of ICP. In some embodiments, the ultrasound is used to determine Doppler flow in the artery and the velocity measurements are used to determine the positions of sample volumes from which measurements can be taken for the assessment of arterial wall and tissue motion. In some embodiments, the arterial wall and tissue motion is determined based on phase shifts in RF echoes from the measurement volume. In some embodiments, the similarity of wave form features includes the ratio of the amplitudes of the diastolic and systolic peaks of the tissue motion waveforms. In some embodiments the measured wall motion is used to identify segments of the ophthalmic artery to be compared for similarity in terms of a metric based on Doppler flow velocity waveform features. In some embodiments the Doppler flow velocity, and visualizations thereof, including color flow Doppler and power Doppler, are used to identify segments of the ophthalmic artery to be compared for similarity in terms of a metric based on waveforms representing motion of the arterial wall and adjacent tissues.

In some embodiments, the measured wall motion is used to identify locations of restricted wall motion due to the proximity to the point of dural penetration, or emergence of the ophthalmic artery from the internal carotid artery. In some embodiments, the tissue motion estimator is a complex cross correlation estimator. In some embodiments, the two or more pressure levels are applied as a continuously-variable pressure characteristic as a function of time. In some embodiments, the pressure in the pressure cuff is adjusted until the IOA waveform and EOA waveform are similar; and estimating ICP of subject based on the pressure applied by the pressure cuff. In some embodiments, the steps of the method include calculating metrics for the IOA and EOA are based on waveform features as a function of applied external pressure, comparing the IOA metrics and EOA metrics, and estimating ICP at pressure at which EOA is closets to the IOA metric.

In some embodiments the method includes using an apparatus for noninvasively measuring intracranial pressure of a subject having an ultrasonic transducer adapted for transmitting an ultrasonic signal into the orbit of the eye, a holder for holding the transducer adapted to stably position the transducer on the subject, an ultrasound transmitter and receiver for transmitting and receiving ultrasonic signals, a pressure applicator adapted for applying external pressure to the extracranial segment of the ophthalmic artery of the subject, a digitizer for digitizing the ultrasonic signal from the ultrasonic receiver, a processor adapted to processing data for estimating the underlying tissue displacement from the ultrasonic signal, and a display for displaying wave forms of tissue displacement.

Other objects of the present invention are achieved by provision of a method for noninvasively measuring intracranial pressure (ICP) of a subject based on comparing the motion of tissues adjacent to an arterial wall, near two or more arterial segments, one segment being substantially subject to, and one substantially not subject to, intracranial pressure.

In some embodiments, the method for noninvasively measuring intracranial pressure (ICP) is based on comparing the motion of the arterial wall in the intracranial and extracranial segments of the opthalmic artery, as well as the Doppler flow velocities in vessel lumina adjacent to the same tissues. In some embodiments, the method for noninvasively measuring intracranial pressure (ICP) of a subject includes the steps of: locating and monitoring, with an ultrasonic transducer, the wall motion of an extracranial ophthalmic artery segment (EOA), and the wall motion of an intracranial ophthalmic artery segment (IOA), acquiring a record of IOA and EOA waveforms at a first pressure. In some embodiments, the EOA is placed under external pressure applied by the pressure cuff the pressure in the pressure cuff is altered to a second pressure, the IOA and EOA waveforms are recorded at the second pressure level, pressure in the pressure cuff is altered to several additional pressure levels, the IOA and EOA waveforms are recorded at each pressure level and ICP of the subject is estimated based on the recorded IOA and EOA waveforms.

Other objects of the invention and its particular features and advantages will become more apparent from consideration of the following drawings and accompanying detailed description.

Measurement of the arterial wall motion using ultrasound is known to the art, as is the relationship between transmural pressure on arterial walls and the compliance of those walls. However, measuring wall motion at two positions along an arterial segment, where one segment is placed under external applied pressure, and adjusting that pressure to until the two waveforms are maximally similar, according to some metrics, is novel.

There are many advantages to the present invention. The present invention results in higher quality signals, in that the detected wave reflections exhibit inherently higher SNR. Improved signal fidelity allows for the detection of lower amplitude waveform features. For example, flow waveform features during diastole, where the flow velocities diminish relative to systole, and where there may be a small amount of reverse flow, are easily buried in noise. Signals with high SNR are, however, much higher above the “noise floor”.

Further, the quality of Doppler signals depends on the location of the Doppler range gate in the vessel. It is dependent on the position of the range gate within the lumen, and also the angle between the insonation line and the flow direction. Higher SNR signals can better tolerate suboptimal range gate location and insonation line selection.

Higher signal quality thus translates, practically, to higher precision of the determined ICP, higher accuracy, higher repeatability, and fewer measurement datasets that must be discarded owing to poor signal quality. Measurements can also be performed faster, since fewer pulses are needed for analysis.

Modulation of the basic shape by the OA depends on the local elastic properties of the OA. When external pressure is applied to the EOA, this changes the local elastic properties, making the vessel wall more elastically compliant. The invention recognizes this is the primary effect of the variable applied pressure used to determine ICP. Changes in flow velocities are secondary to this change in the local mechanical properties of the wall. The OA possesses no mechanism to influence the Doppler flow velocities in its lumen except via changes in the effective material properties of its wall. In general, it is preferred to measure a primary affected property rather than a secondary, derived, property.

Also, the invention recognizes the motion of the arterial wall leads to motion of surrounding tissue. This means that the “tissue Doppler” signal may be obtained also from surrounding tissue. This makes it easier to position the transducer and range gates in order to acquire the required signals.

Another advantage is measurement of a primary quantity rather than a secondary quantity. The basic shape of the Doppler waveform in the OA is determined by the systolic ejection of the heart, as well as subsequent propagation and reflection of pressure waves through the circulatory system.

### BRIEF DESCRIPTION OF THE DRAWINGS

FIG. 1 is an illustration of how system blood pressure can be measured using the prior art oscillometric method.

FIG. 2(a) is an illustration of an M-mode matrix for 0 mmHg.

FIG. 2(b) is an illustration of a distension wave form for 0 mmHg.

FIG. 2(c) is an illustration of an M-mode matrix for 32 mmHg.

FIG. 2(d) is an illustration of a distension wave form for 32 mmHg.

FIG. 3(a) is an illustration of an M-mode matrix for 4 mmHg.

FIG. 3(b) is an illustration of a distension wave form for 4 mmHg

FIG. 4(a) is an illustration of an M-mode matrix for 16 mmHg.

FIG. 4(b) is an illustration of a distension wave form for 16 mmHg

FIG. 5(a) is an illustration of an M-mode matrix for 28 mmHg.

FIG. 5(b) is an illustration of a distension wave form for 28 mmHg

FIG. 6 is a chart showing ratio of diastolic to systolic peak amplitudes measured in extracranial ophthalmic artery.

FIG. 7 is a chart showing ratio of diastolic to systolic peak amplitudes measured in intracranial and extracranial ophthalmic artery.

FIG. 8(a) is an illustration of an M-mode matrix for 0 mmHg.

FIG. 8(b) is an illustration of a distension wave form for 0 mmHg.

FIG. 9 is a chart showing a measurement-based model of the luminal area of the brachial artery as a function of transmural pressure.

FIG. 10(a) is an illustration of the intracranial pressure measurement device on a patient.

FIG. 10(b) is an illustration of the monitor and display of the intracranial pressure measurement device.

### DETAILED DESCRIPTION OF THE INVENTION

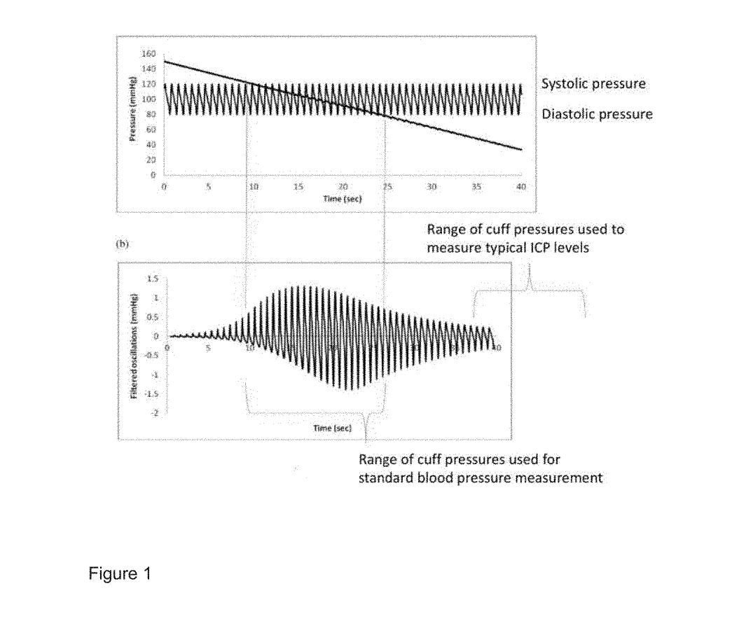

FIG. 1 illustrates how systemic blood pressure is measured via the common oscillometric method. FIG. 1 adapted from C. F. Babbs, “Oscillometric measurement of systolic and diastolic blood pressures validated in a physiologic mathematical model,” Biomedical engineering online, vol. 11, no. 1, p. 1, 2012.

The illustration of FIG. 1 describes how systemic blood pressure is measured by the oscillometric method. When the cuff pressure exceeds systolic pressure, negligible pulsation is observed superimposed on the cuff pressure. As the cuff is deflated, pulsation amplitude rapidly increases until, when the cuff pressure is close to mean arterial pressure, a maximum is observed. With further decreases in cuff pressure, the pulse amplitude decreases monotonically. Thus, below diastolic pressure, there is a one-to-one mapping between pulse amplitude and cuff pressure. Since ICP is always less than diastolic pressure, it is possible to measure it unambiguously by measuring pulsation amplitude.

In the method, the amplitudes of blood pressure pulses are measured as perturbations of the pressure in an external cuff that applies pressure to the limb. Between systolic and diastolic pressures, the pulse amplitude distribution is non-monotonic, which means there is no one-to-one mapping between cuff pressure and pulse amplitude. (A monotonic function is one that either increases or decreases, but does not both increase and decrease. Between systolic and diastolic pressures, the pulse amplitudes first increase, and then decrease. This is a demonstration of non-monotonic behavior. At pressures lower than diastolic pressure, the amplitudes of the pulses decrease as the externally-applied pressure falls, which is a demonstration of monotonically decreasing behavior.)

Below diastolic pressure, the pulse amplitude decreases monotonically with pressure, meaning that there exists a one-to-one mapping between pulse amplitude and applied cuff pressure. The one-to-one mapping means that, given the measured amplitude of a particular pulse, it is possible to unambiguously “look-up” the value of the externally-applied pressure that prevailed during that pulse.

In the application of non-invasive ICP measurement, we determine the ICP by matching features of the waveforms obtained in the EOA and IOA. The non-invasive ICP measurement is taken to be equal to that externally-applied pressure for which a similarity metric determines maximal similarity between the EOA and IOA waveforms. The one-to-one mapping means that if we apply an external pressure to the EOA that matches that applied to the IOA (which is the ICP), we can unambiguously determine that the pressure we apply is equal to ICP, under the assumption of similar material properties of the IOA and EOA. There is the possibility that tapering of the vessel could sufficiently differentially affect the material properties of the EOA and IOA so as to bias the similarity metric to be maximum at a point where the externally-applied pressure and true ICP are not equal. It is also possible that, owing to the prolonged exposure of the IOA to ICP, the material properties of the wall of that segment differ substantially from those of the EOA, which was not exposed to the ICP over the lifetime of the individual. Since normal ICP is much lower than BP, it is likely that any such effect is small.

Measurement of the arterial wall motion using ultrasound is known to the art, as is the relationship between transmural pressure on arterial walls and the compliance of those walls. However, measuring wall motion at two positions along an arterial segment, where one segment is placed under external applied pressure, and adjusting that external pressure to until the two waveforms, are maximally similar, according to some metrics, is novel.

The one-to-one correspondence is not useful for standard BP measurement, since systemic BP lies between diastolic and systolic pressures, for which this property does not hold. This property has not been previously used to measure ICP.

The invention assumes that the walls of the ophthalmic artery and peripheral arteries (e.g., brachial, radial and femoral) behave similarly when external pressure is applied. This assumption is strongly supported by the observations of applied-pressure-dependent pulsation amplitudes observed in the central retinal artery (CRA, which branches from the ophthalmic artery) during opthalmodynamometry, as described in R. Stodtmeister, T. Oppitz, E. Spoerl, M. Haustein, and A. G. Boehm, “Contact lens dynamometry: the influence of age,” Investigative ophthalmology & visual science, vol. 51, no. 12, pp. 6620-6624, 2010.

Methods for tracking the motion of arterial walls using non-invasive ultrasound were first described in the 1970s as describe by D. E. Hokanson, D. J. Mozersky, D. S. Sumner, and D. E. Strandness, “A phase-locked echo tracking system for recording arterial diameter changes in vivo.,” Journal of Applied Physiology, vol. 32, no. 5, pp. 728-733, 1972.

Since flow velocities in both the IOA and EOA can be measured using Doppler ultrasound, it is possible to also measure the arterial wall motion. This requires the ultrasound system to be operated without the “wall filter” used to remove the wall motion artifact from the (much lower power) Doppler flow signal. Such measurement of wall motion, for the purposes of observing a pressure balance condition for ICP measurement, is not known in the prior art.

What is needed is a device and method wherein sensitivity to the condition of equalization can be improved by, rather than comparing flow velocities in the EOA and IOA, comparing the arterial wall motion in these arterial segments. Further what is needed a method that requires less precision in the positioning of transducers and the Doppler range gates.

### Example 1

EOA measurements were taken in a subject. Clinical data were acquired using a 2 MHz single channel ultrasound system from the EOA of a subject. The external pressure was increased from 0 mmHg to 32 mmHg in steps of 4 mmHg. The M-mode matrix (ultrasound reflection amplitude-vs-depth, as a function of time) was processed using a complex correlation model to produce wall distension waveforms as described in P. J. Brands, A. P. G. Hoeks, L. A. F. Ledoux, and R. S. Reneman, “A radio frequency domain complex cross-correlation model to estimate blood flow velocity and tissue motion by means of ultrasound,” Ultrasound in Medicine & Biology, vol. 23, pp. 911-920, 1997 (“Brands”). This matrix contains as columns the raw RF echoes and differs from standard ultrasound M-mode in that the source signals are not demodulated).

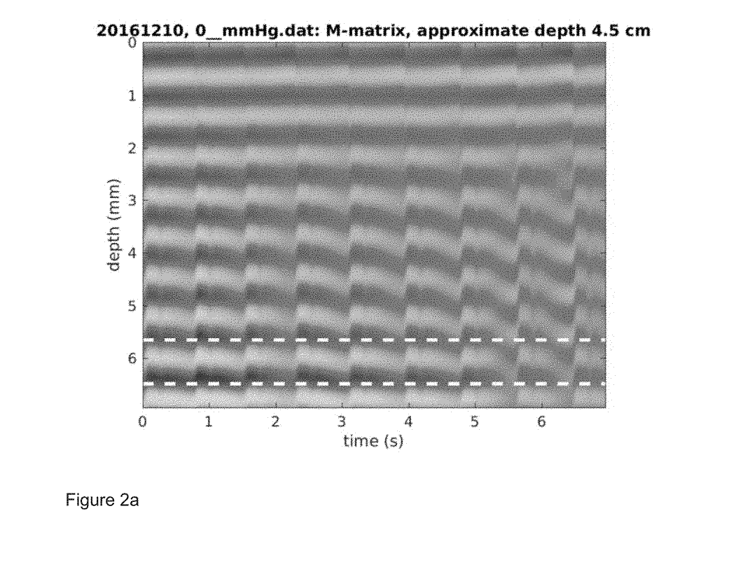

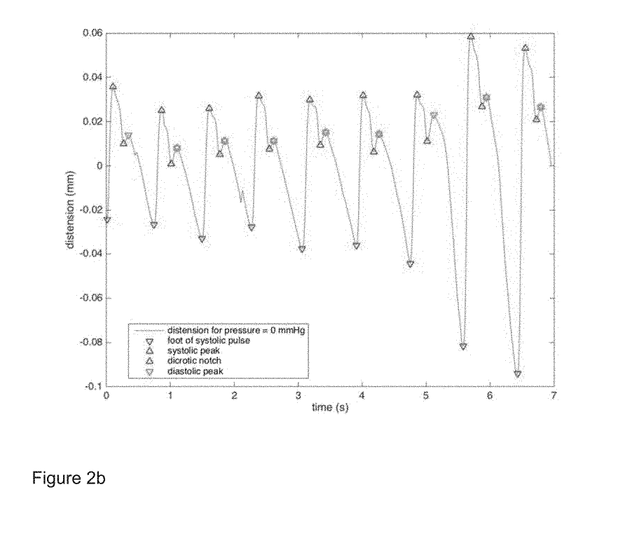

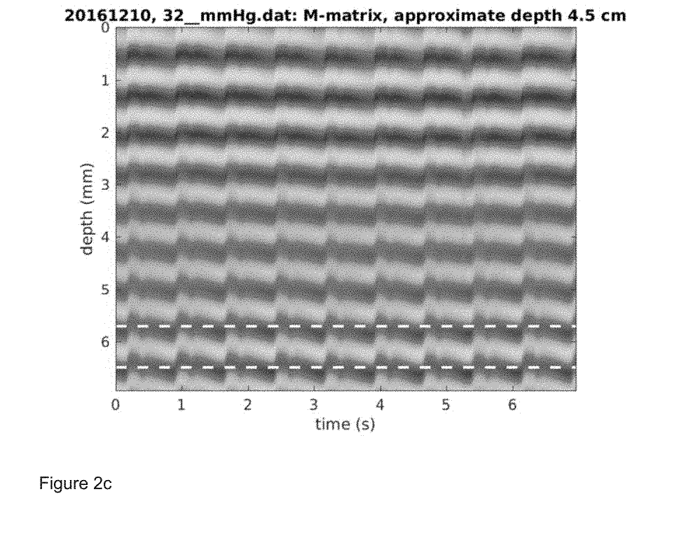

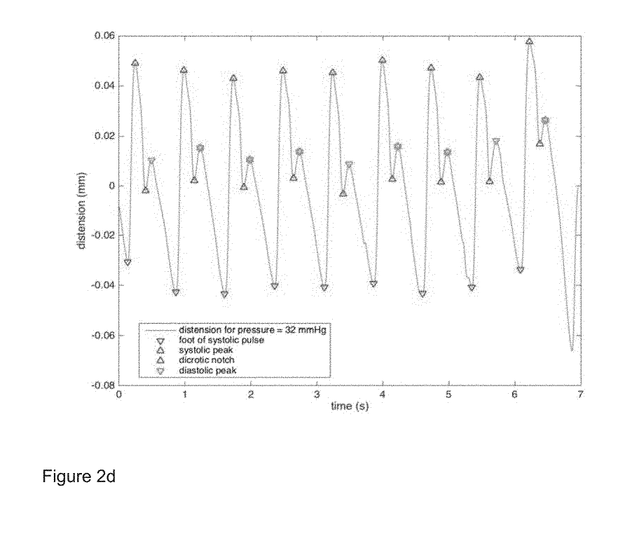

FIGS. 2(a) and (b) show the M-mode matrix and the distension waveform for 0 mmHg of applied external pressure to the orbit of the eye and FIGS. 2(c) and (d) show the M-mode matrix and the distension waveform for 32 mmHg of applied external pressure to the orbit of the eye. These signals are obtained from the EOA. Ultrasound clutter, due to reverberations and reflections, is present in the M-mode matrices. The dotted white lines shown in all the Figures are the depth limits of the data used to generate the distension curves. Only pulses having green markers were included in calculations. Others are considered outliers.

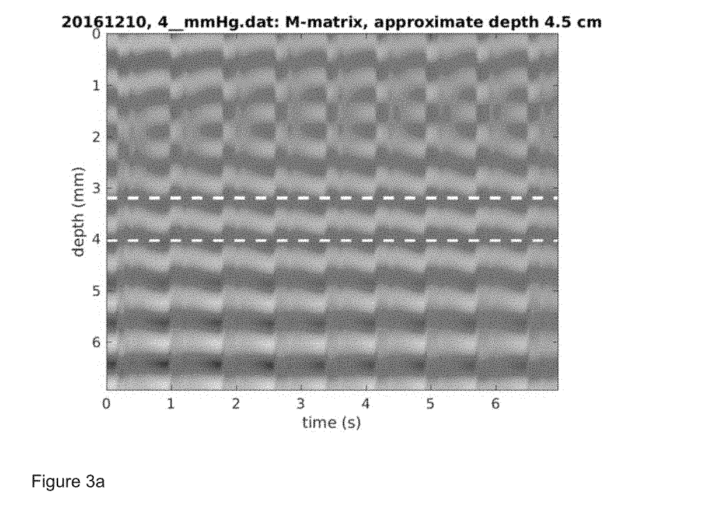

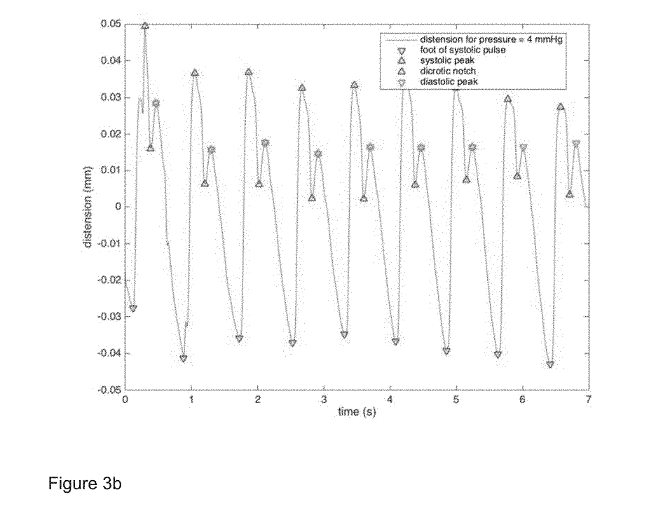

FIGS. 3(a) and 3(b) show the M-mode matrix and distension waveform respectively for 4 mmHg of applied external pressure to the orbit of the eye.

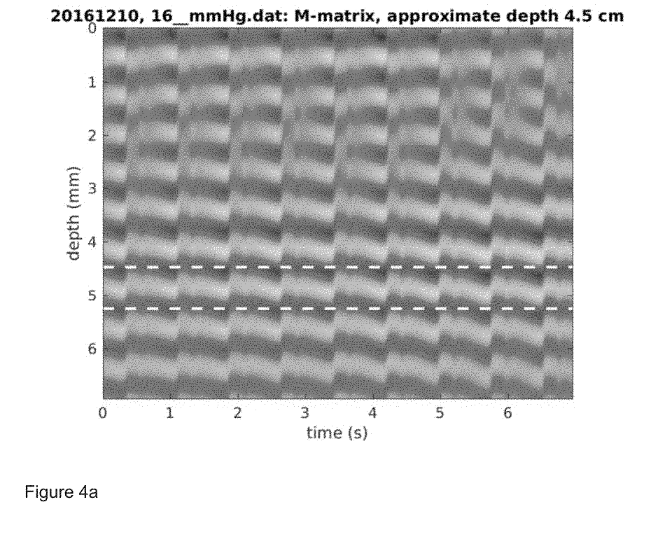

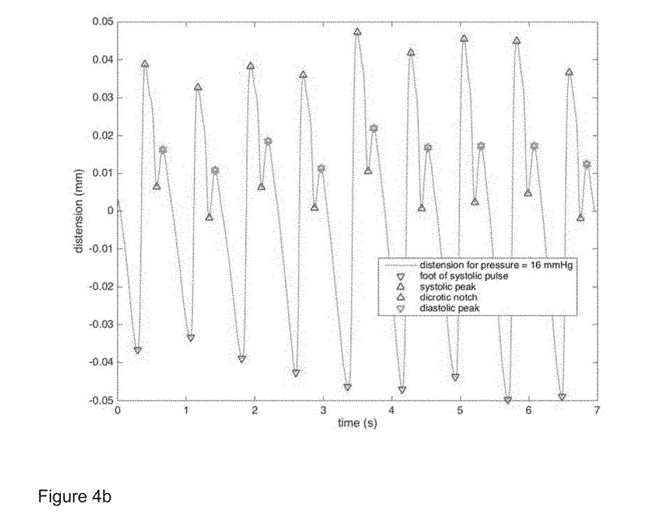

FIGS. 4(a) and 4(b) show the M-mode matrix and distension waveform respectively for 16 mmHg of applied external pressure to the orbit of the eye.

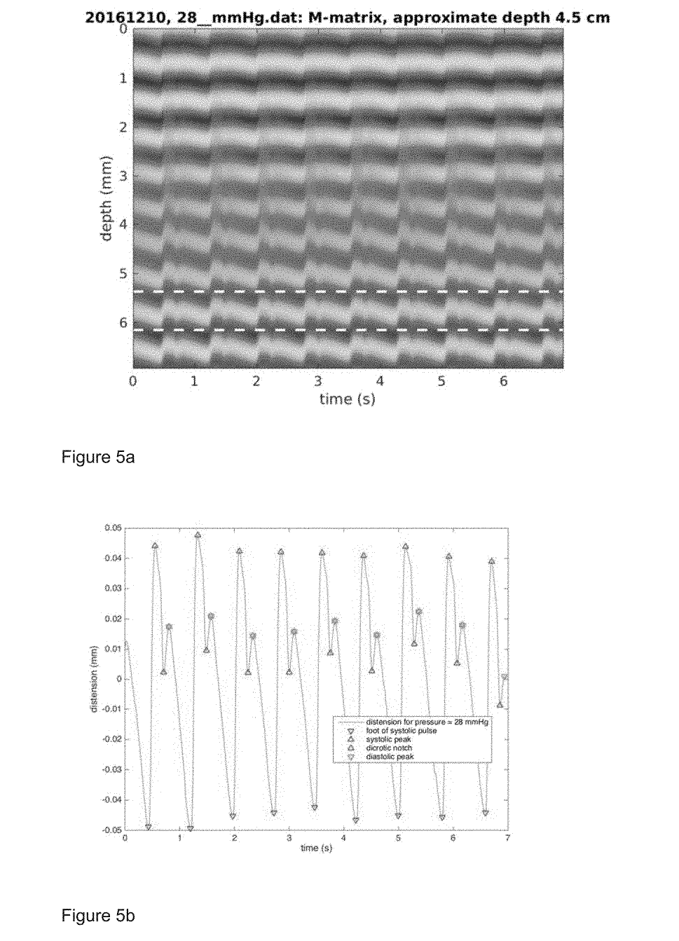

FIGS. 5(a) and 5(b) show the M-mode matrix and distension waveform respectively for 28 mmHg of applied external pressure to the orbit of the eye.

In all cases, the distension waveform contains two peaks: a larger systolic peak, followed later by a smaller peak corresponding to summed reflections of the systolic peak within the circulatory system. We term this the “diastolic peak”, since it occurs during the diastolic phase of the cardiac cycle. It is clear that, as the applied external pressure increases, the relative height of the diastolic peak to the systolic peak becomes smaller.

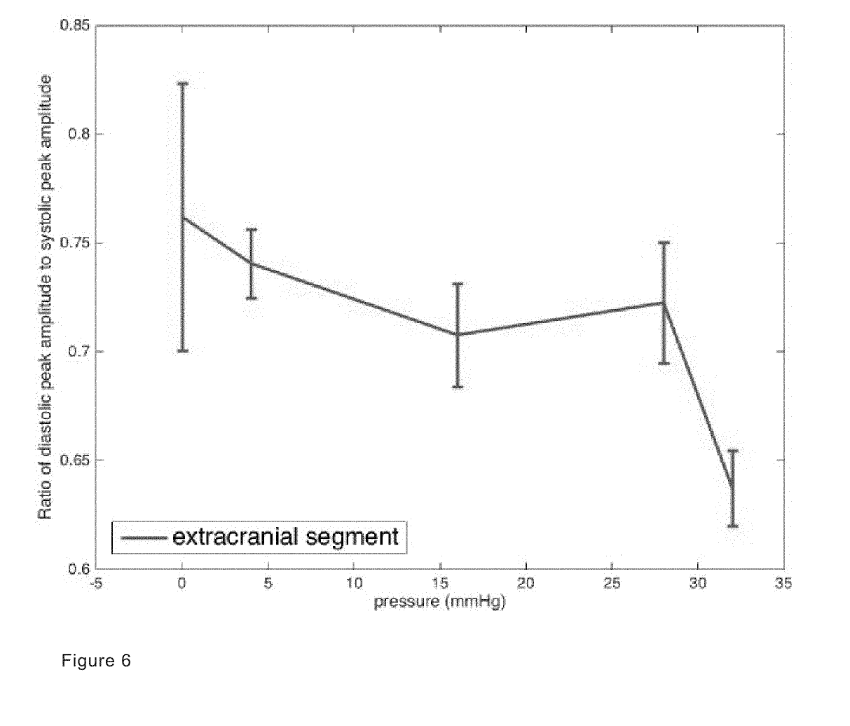

In FIG. 6 the graph shows the EOA of the first subject, and how the ratio between the amplitudes of the diastolic and systolic pulses tends to fall as more pressure is applied to the orbit of the eye. The ratio of diastolic to systolic peak heights tends to fall as external pressure on the orbit is increased. The amplitude of the diastolic peak is taken as the maximum diastolic peak value minus the systolic foot value.

Auto-segmentation was applied to the distension waveforms to identify the systolic foot and peak, as well as the diastolic foot (dicrotic notch) and peak. Outliers were rejected by including only pulses having diastolic/systolic ratios within the central two interquartile ranges ±half of the summed spans of the central two interquartile ranges.

### Example 2

EOA and IOA measurements were taken in a second subject. The data of the Example 1 in FIGS. 2-6, suggests that the ratio of the diastolic to systolic peak amplitudes is a convenient, overall-amplitude-normalized metric that is dependent on applied pressure. The absolute pulse amplitude should be a viable measure in many cases. However, the data from Example 1 illustrates how the measured amplitude can be sensitive to, for example, the angle between the beam and the vessel, the position of the range rate within the tissue, and reverberations [clutter] in the tissue. The invention contemplates a preferred embodiment using a self-normalized metric.

To gain further data and explore the peak ratio metric, the inventors performed further measurements in the EOA and IOA of a second subject.

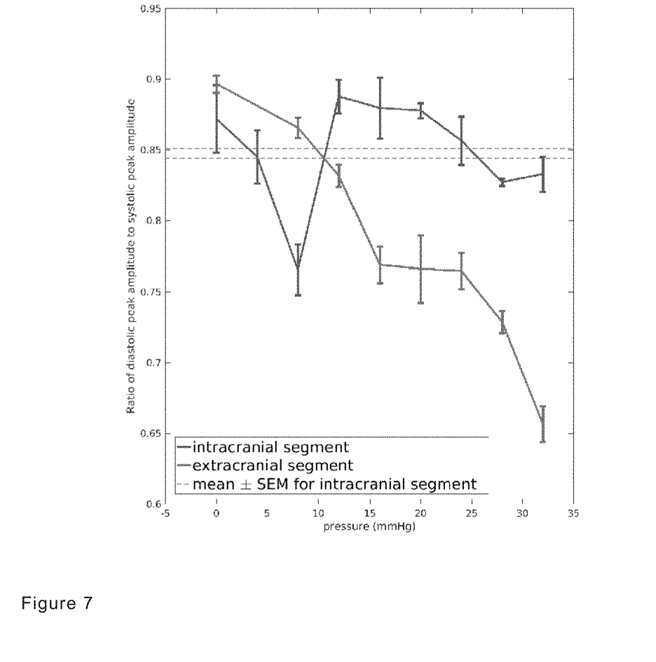

FIG. 7 is a chart showing the ratio of diastolic to systolic peak amplitudes measured in intracranial and extracranial ophthalmic artery of the second subject. This chart shows that the diastolic to systolic peak ratio falls (in the EOA) as a function of applied external pressure, but not in the IOA. Based on this curve, it is likely that the ICP is less than 15 mmHg, since this is the region where the metric is most similar between the EOA and IOA segments.

The ratio between the amplitudes of the diastolic and systolic pulses tends to fall as more pressure is applied to the orbit. However, this trend is not present in the IOA, which is presumed to be subject to ICP, and not the external applied pressure. The chart is a real-world demonstration of the method in which there are many sources of measurement uncertainty. Under ideal circumstances, the IOA curve would be a constant value (independent of applied pressure). The EOA curve would be a monotonically decreasing function on applied pressure. The point at which the EOA curve crosses the IOA curve would represent the estimate of ICP. (Data points represent mean values ±standard error of the mean [SEM]. The IOA value at 8 mmHg is a likely outlier.)

3.3 Measurements in Third Subject with Range Gate Outside OA Lumen

### Example 3

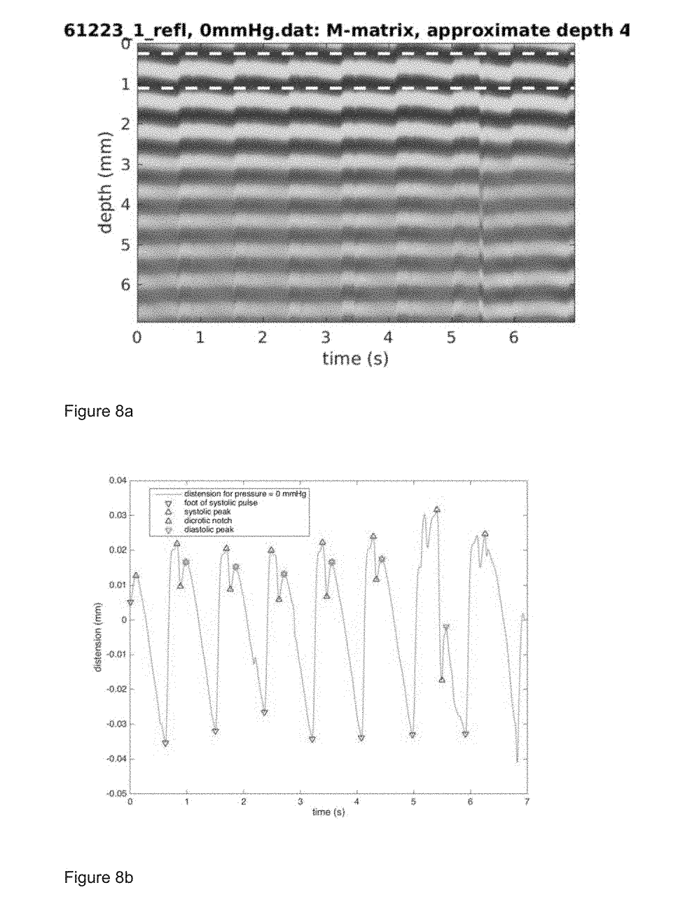

Measurements were taken in a third subject using a range gate outside the OA lumen. To illustrate that it is possible to obtain potentially useful wall distension waveforms without precise positioning of the Doppler range gate within the OA lumen, the range gate was deliberately displaced so that no Doppler flow signal could be observed. FIG. 8(a) is an illustration of an M-mode matrix for 0 mmHg. FIG. 8(b) is an illustration of a distension wave form for 0 mmHg generated when using a range gate outside the OA lumen.

FIGS. 8(a) and 8(b) show that physiologically reasonable distension waveforms can be measured even though the range gate is not situated over, or even immediately adjacent to, the arterial lumen. It seems that motion of tissues nearby the OA, and ultrasound clutter, can make it easier to find useful signals, especially when a non-imaging ultrasound system is used to acquire the waveforms.

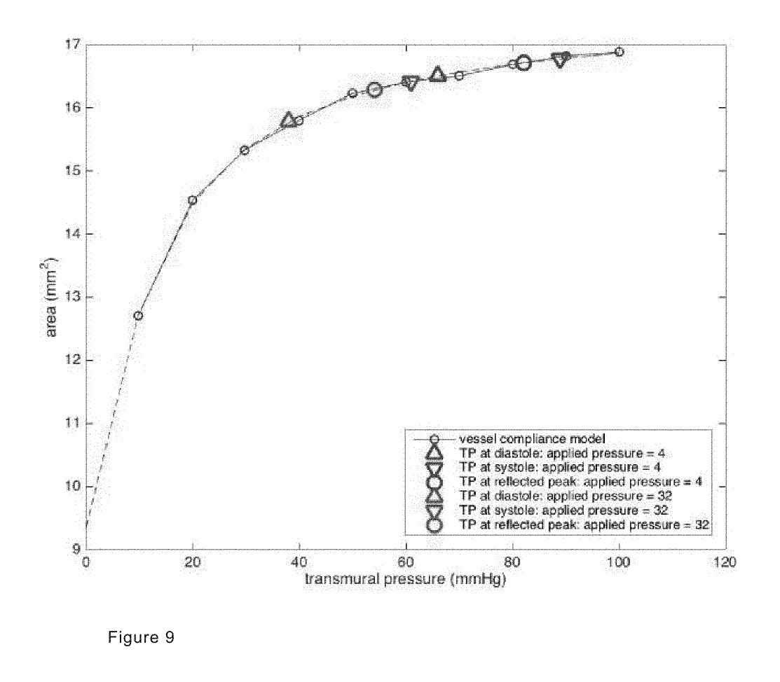

FIG. 9 shows a measurement-based model of the luminal area of the brachial artery as a function of transmural pressure, as presented in A. Bank, D. Kaiser, S. Rajala, and A. Cheng, “In vivo human brachial artery elastic mechanics: Effects of smooth muscle relaxation,” Circulation, vol. 100, pp. 41-7, 1999. The assumption is made that the systolic blood pressure (in the OA) is 93 mmHg and the diastolic blood pressure is 70 mmHg. Operating points for external applied pressures of 4 mmHg and 32 mmHg are considered. The upright blue triangle represents the operating point at diastole for 4 mmHg of applied pressure, while the blue circle indicates that for the corresponding systole. The difference in area between the latter and the former is proportional to the vessel distention across the cardiac cycle. The red symbols represent the respective areas when the applied external pressure is 32 mmHg. The diastolic:systolic peak ratios are 0.76 and 0.78 for 4 mmHg and 32 mmHg external pressures, respectively. The effect predicted by the model is thus opposite to that we observe in the EOA

As FIG. 9 explains, the observed phenomenon of diastolic:systolic ratio decrease as a function of increasing external pressure is not consistent with current knowledge of the behavior of the brachial artery. The measurement-based model presented in A. Bank, D. Kaiser, S. Rajala, and A. Cheng, “In vivo human brachial artery elastic mechanics: Effects of smooth muscle relaxation,” Circulation, vol. 100, pp. 41-7, 1999, predicts that this ratio should increase will increasing applied pressure, while the data demonstrates observed decreases in two subjects.

It is thus not obvious from studies of the brachial artery that one could predict the observed behavior of the EOA under applied external pressure.

In Examples 1-3, the measurement volumes within with tissue were selected based on proximity to pulsatile flow detected by Doppler flow velocity measurement. In cases such as where the signal-to-noise ratio is low (e.g., at low Doppler insonation power) there may be advantage in using tissue motion measurements to select measurement volumes for Doppler-flow-based non-invasive ICP determination according to methods such as those taught in '477.

Also, it is possible that tissue motion may identify arterial segments that are unsuitable for selection as measurement volumes for Doppler-flow-based methods. For example, at the point at which the opthalmic artery crosses the dura, the measurement volume may include parts of both the IOA and EOA. The dura, being relatively stiff, reduces the ability of the arterial wall and adjacent tissues to distend in response to changes in pressure and volume. The decreased wall motion at this location may be used to identify the location of the dural penetration of the OA, so that this location may be avoided as a measurement volume. Similarly, at the emergence of the OA from the ICA, the attachment of the OA to the ICA may restrict wall motion, or distort this motion. Measurement of wall motion as taught in this application can determine whether a selected Doppler flow measurement volume is unsuitable for the method of '477.

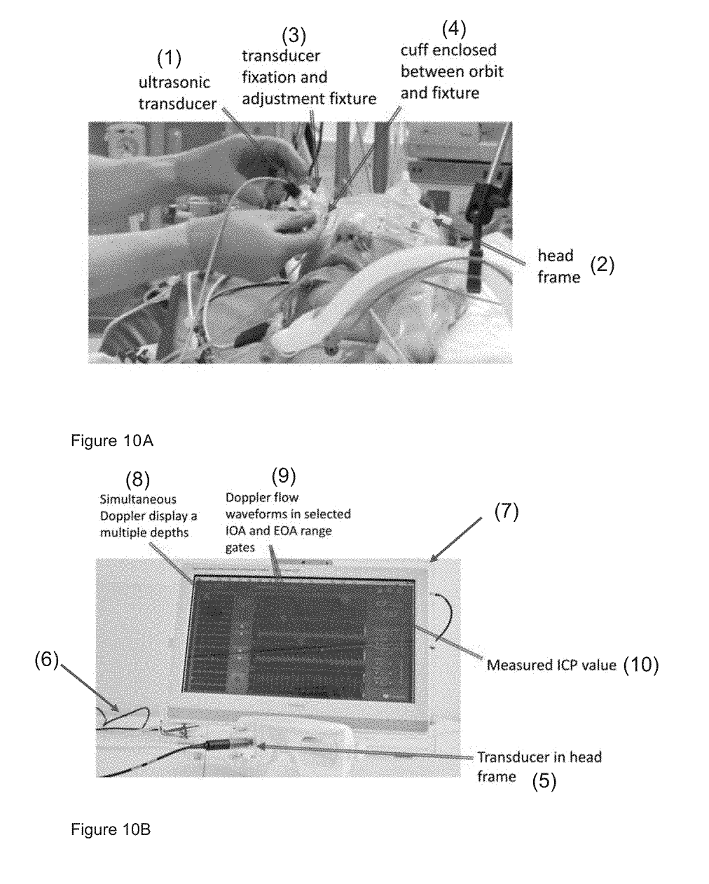

An illustration of an embodiment of the invention can be seen FIGS. 10 (a) and (b).

FIG. 10 (a) shows a transducer (1) positioned in the head frame (2) and secured in an adjustable fixture (3). An inflatable annular cuff (4) is enclosed between the fixture and the orbit of the eye of the human subject. The ultrasonic transducer, transducer fixation and adjustment fixture and cuff are all held in place by the head frame (2). FIG. 10 (b) shows how the transducer in the head frame (5) is electrically connected to a processor (6), a modified Vittamed 205 device, via a cord (6). The processor is configured to extract the raw RF echoes and synchronization signals so that the wall motion signals can be generated (modification not shown). The processor is connected to a screen display (7). The display is configured to show simultaneous Doppler display at multiple depths (8), Doppler flow waveforms in selected IOA and EOA range gates (9) and measured ICP value (10). The multidepth Doppler flow waveforms are displayed on the screen and are used to aid in the location of the range gates for wall motion estimation.

One embodiment of the invention has the following components. An ultrasonic transducer which can be of single crystal type (non-imaging) or array type. Typically, the range of frequencies useful for performing measurements on the ophthalmic artery is 1-18 MHz. In the examples provided the inventors used a frequency of 2 MHz, which realizes good penetration at the low power levels acceptable for transorbital ultrasound, but at the expense of poorer spatial resolution. Coded excitation of the transducer can help reduce the power required to achieve a given SNR, and hence allow the use of higher frequencies as described in M. H. Pedersen, T. X. Misaridis, and J. A. Jensen, “Clinical evaluation of chirp-coded excitation in medical ultrasound,” Ultrasound in Medicine & Biology, vol. 29, no. 6, pp. 895-905, 2003. (Higher frequencies offer better resolution, but are more highly attenuated by tissue, and so have poorer tissue penetration.) Similarly, plane wave ultrasound imaging can also achieve better SNR for a given ultrasound power level as described in R. Urs, J. A. Ketterling, and R. H. Silverman, “Ultrafast ultrasound imaging of ocular anatomy and blood flow,” Investigative Ophthalmology & Visual Science, vol. 57, no. 8, pp. 3810-3816, 2016 (“Urs”).

It is likely, using coded or plane wave imaging, that a frequency of at least 10 MHz could be used without exceeding guidelines for transorbital imaging.

A transducer fixation and adjustment fixture for holding the transducer is also included in the embodiment. This transducer holder allows the transducer to be adjusted but also to be stably positioned during the examination and measurement. The transducer, so positioned, is able to interrogate the IOA, EOA, internal carotid artery (ICA) and surrounding tissues.

An acoustical couple for acoustically-coupling the transducer with the tissue of the subject. Typically, this means applying a gel between the transducer face and the eyelid.

A headframe for holding the transducer and transducer holder.

An ultrasound transmitter and receiver for transmitting and receiving ultrasound signals.

A pressure applicator for applying external pressure to the EOA. Typically, this is accomplished by means of an air-filled cuff that is placed between the orbit of the eye and the head-frame. The cuff may have an annular shape so that the transducer may be situated within the aperture of the annulus. Liquid- or gel-filled cuffs may also be used, if care is taken to account for any pressure these might apply to the orbit owning to their weight.

A digitizer for digitizing or otherwise storing the RF echo signal received by the receiver. The digitizer can be a processor, part of a processor or other hardware, software combination.

A processor for processing data for estimating the underlying tissue displacement from the RF echo records. Typically, this is achieved by measuring the phase shifts of the RF signal that occur over time within a range of depths where the tissue of interest is encountered. (This range of depths is termed the “range gate”.) These phase shifts are converted into velocity estimates, and then integrated over time to produce the wall distension waveform.

The method for noninvasively measuring ICP includes the following steps;

1. Subject rests in supine position.

2. Ultrasound gel is applied to an eyelid.

3. A freehand pilot scan is performed to determine approximate transducer location. The Doppler flow and/or wall motion are monitored in real-time, while the operator optimizes the transducer position and position of the range gates for the ICA, IOA and EOA. The IOA range gate is determined based on the characteristics of its Doppler flow waveform and proximity to the ICA (which itself has a characteristic Doppler flow waveform typical of a large conduit artery). If imaging is available, the image, color flow map, and flow power flow map may be used to guide positioning of the range gates.

4. The head-frame is placed on the head of the subject, and the pressure cuff is inserted between the frame and the orbit of the eye. The transducer is placed within the central cavity of the annular cuff. Gel is applied between the transducer and the eyelid. FIG. 10 shows the head frame secured to the head of a patient, with the transducer placed within the fixation device. It also shows the device to which the transducer is connected.

5. The ICA, EOA and IOA wall motion (and, optionally, Doppler flow signals) are located and continuously monitored.

6. After a record of the IOA and EOA waveforms is acquired at the current applied pressure (0 mmHg), the pressure is set to several different higher pressure levels.

7. At each pressure, the operator ensures the signal quality remains acceptable. Small adjustments of transducer position, or position of the range gates, may be required. The waveforms are recorded at each higher pressure.

8. Based on the IOA and EOA waveforms recorded at all pressures, the ICP is estimated:
(a) Each recorded RF echo is assembled as a column of the matrix M, the RF “M-mode” matrix. Specific matrix examples are shown in the FIGS. 2a and 2c. Each column is a time series, where increasing time (echo arrival time) is proportional to the depth at which the reflection occurred. Generally, a separate M matrix will be formed from echoes that include the IOA and EOA range gates.
(b) Each range gate may be adjusted, either manually or automatically, to include a sub-range of depths. Also, several sub-range gates may be defined for each M. For example, in FIG. 2, single sub-range gates per M, which happen to correspond to approximately one period of the RF carrier.
(c) An algorithm is applied to each sub-range gate to estimate the motion of the tissue/blood located within in the range gate. The embodiment of the processing method used to produce the distension waveforms has been described previously in the specification. In “ultra-fast Doppler” modes as disclosed in Urs, tissue and blood Doppler signals are available for all points in the ultrasound field-of-view for all sample times (this is because, when plane wave imaging is used, ultrasound focusing is performed only in receive mode). Retrospective analysis of these Doppler waveforms can be used to refine the final IOA and EOA range gate positions. This is especially helpful if inflation/deflation of the cuff moves the artery and hence the optimal range gate positions.
(d) The algorithm yields velocity estimates for the tissue/blood. Velocities are proportional to the phase shifts of the received signal between ultrasound echoes (columns of the matrix M).
(e) The velocity estimates are integrated over time to yield displacement (distension) estimates.
(f) Automatic and/or manual processing is used to identify the foot and peak timing and magnitude of each systolic pulse, the dichroitic notch, and the diastolic peak.
(g) Based on waveform features, metrics are calculated. Examples include:
   i. systolic peak amplitude
   ii. diastolic peak amplitude
   iii. ratio of diastolic to systolic peak amplitude
   iv. delay between systolic peak and dicrotic notch
   v. delay between systolic foot and dicrotic notch
   vi. delay between systolic peak and dicrotic notch
   vii. delay between dicrotic notch and diastolic peak
   viii. pulse period
(h) The timing metrics may be useful in assessing the systemic vascular tone changes and blood pressure modulation due to factors such as respiration, which also modulate ICP.

(i) The metrics, as a function of applied external pressure, are compared for the IOA and EOA. The point at which the EOA metric value matches the IOA metric is one estimate for the ICP. In general, by analyzing the values and variations (uncertainties) in the IOA and EOA metrics, statistical methods can be used to determine, to a given degree of confidence, the pressure at which the EOA metric is closest to the IOA metric, thus providing the basis for an ICP estimate.

The Processing method for obtaining wall motion estimates is as follows. RF line processing to yield wall distension estimates is as follows:

1. Data are acquired using a 2 MHz single channel ultrasound system (Vittamed 205).

2. The RF signals are digitized at 400 MHz at 14-bit resolution. (ADQ214, Signal Processing Devices Sweden AB, Linkoping, Sweden.)

3. An “M-matrix” is assembled from the recorded RF of successive pulses. (This is not a standard M-mode matrix, since no demodulation is performed.)

4. The voltage vs A/D value amplitude characteristic of the hardware is applied to the recorded A/D values to yield voltage values. FIG. 2 shows the (voltage) M-matrices for the cases of 0 mmHg and 32 mmHg of applied external pressure.

5. The depth range is now selected to include the arterial wall. This is done based on the operator's (one of skill in the art) experience and judgment. At many depths, there are time-dependent artifacts, and signals that are not consistent with arterial wall motion. Each depth range is selected to include approximately one period of the RF carrier.

6. The selected rows of the M-matrix are fed into the complex correlation model-based estimator (C3M) proposed in Brands. This is implemented easily in Matlab as:

```
function phi = c3mfn(MT, wallInd);
% MT is transpose of std M mode
[il,kl] = size(MT);
for n = 1:il−1
  ic = detrend(MT(n,wallInd));
  id = detrend(MT(n+1,wallInd)); %lag 1 on time axis
  ie = detrend(MT(n,wallInd+1)); %lag 1 on spatial axis
  hic = hilbert(ic);
  hid = hilbert(id);
  hie = hilbert(ie);
  R01 = hic*hid′;
  R10 = hic*hie′;
  argR01 = angle(R01);
  argR10 = angle(R10);
  phi(n) − argR01/argR10;
end
```

7. These velocity shifts are integrated in time to produce distension estimates, examples of which also appear in FIG. 2. (A 6th-order Butterworth low-pass filter with 25 Hz cutoff frequency is applied to the distension waveforms).

8. Auto-segmentation is applied to the distension waveforms to identify the systolic foot and peak, as well as the diastolic foot (dicrotic notch) and peak. Outliers are rejected by including only pulses having diastolic/systolic ratios within the central two interquartile ranges ±half of the summed spans of the central two interquartile ranges.

Although the invention has been described with reference to a particular arrangement of parts, features and the like, these are not intended to exhaust all possible arrangements or features, and indeed many modifications and variations will be ascertainable to those of skill in the art.

What is claimed is:

1. A method for noninvasively measuring intracranial pressure (ICP) of a subject based on comparing the motion of the arterial wall, in two or more arterial segments, one segment being substantially subject to, and one substantially not subject to, intracranial pressure.

2. A method for noninvasively measuring intracranial pressure (ICP) of a subject based on comparing the motion of tissues adjacent to an arterial wall, near two or more arterial segments, one segment being substantially subject to, and one substantially not subject to, intracranial pressure.

3. The method of claim 1 in which ICP is measured comparing the motion of the arterial wall and adjacent tissues.

4. The method of claim 3 in which motion of the arterial wall and adjacent tissues is assessed using ultrasound.

5. The method of claim 3 in which ultrasound signals are used to determine wall motion waveform features derived from at least two segments of an artery, and in which the similarity of wall motion waveform features, between waveforms derived from the at least two segments of the artery, as a function of external pressure applied to the orbit of the eye of subject, determines the estimate of ICP.

6. The method of claim 1 in which ultrasound is used to determine Doppler flow in the artery and the velocity measurements are used to determine the positions of sample volumes from which measurements can be taken for the assessment of arterial wall and tissue motion.

7. The method of claim 1 in which arterial wall and tissue motion is determined based on phase shifts in RF echoes from the measurement volume.

8. The method of claim 5 in which the similarity of wave form features includes the ratio of the amplitudes of the diastolic and systolic peaks of the tissue motion waveforms.

9. A method for noninvasively measuring intracranial pressure (ICP) based on comparing the motion of the arterial wall in the intracranial and extracranial segments of an artery, as well as the Doppler flow velocities in lumina adjacent to the same tissues.

10. The method of claim 9 in which the measured wall motion is used to identify segments of the artery to be compared for similarity in terms of a metric based on Doppler flow velocity waveform features.

11. The method of claim 9 in which the measured wall motion is used to identify locations of restricted wall motion due to the proximity to the point of dural penetration, or emergence of the ophthalmic artery from the internal carotid artery.

12. The method of claim 9 in which Doppler flow velocity, and visualizations thereof, including color flow Doppler and power Doppler, are used to identify segments of the artery to be compared for similarity in terms of a metric based on waveforms representing motion of the arterial wall and adjacent tissues.

13. The method of claim 1 in which the motion is estimated using a complex cross correlation estimator.

14. A method for noninvasively measuring intracranial pressure (ICP) of a subject comprising the steps of:
locating and monitoring, with an ultrasonic transducer, the wall motion of an extracranial ophthalmic artery segment (EOA), and the wall motion of an intracranial ophthalmic artery segment (IOA);
acquiring a record of IOA and EOA waveforms at a first pressure, wherein said EOA is placed under external pressure applied by the pressure cuff;
altering the pressure in the pressure cuff to a second pressure, recording IOA and EOA waveforms at said second pressure level;
altering the pressure in the pressure cuff to several additional pressure levels;
recording IOA and EOA waveforms at each pressure level; and
estimating ICP of the subject based on recorded IOA and EOA waveforms.

15. The method of claim 14 in which the two or more pressure levels are applied as a continuously-variable pressure characteristic as a function of time.

16. The method of claim 15 further comprising:
adjusting the pressure in the pressure cuff until the IOA waveform and EOA waveform are similar; and
estimating ICP of subject based on the pressure applied by the pressure cuff.

17. The method of claim 14 further comprising:
calculating metrics for the IOA and EOA based on waveform features as a function of applied external pressure;
comparing the IOA metrics and EOA metrics; and
estimating ICP at pressure at which EOA is closets to the IOA metric.

18. The method of claim 14 using an apparatus for noninvasively measuring intracranial pressure of a subject comprising:
an ultrasonic transducer adapted for transmitting an ultrasonic signal into the orbit of the eye;
a holder for holding the transducer adapted to stably position the transducer on the subject;
an ultrasound transmitter and receiver for transmitting and receiving ultrasonic signals;
a pressure applicator adapted for applying external pressure to the extracranial segment of the ophthalmic artery of the subject;
a digitizer for digitizing the ultrasonic signal from the ultrasonic receiver;
a processor adapted to processing data for estimating the underlying tissue displacement from the ultrasonic signal; and
a display for displaying wave forms of tissue displacement.
